(19) United States
(12) Patent Application Publication
(10) Pub. No.: US 2022/0323253 A1
(43) Pub. Date: Oct. 13, 2022

(54) RESONANT NON-SINUSOIDAL DRIVE SIGNALS FOR PHACOEMULSIFICATION SURGERY DEVICES

(71) Applicant: Bausch & Lomb Incorporated, Rochester, NY (US)

(72) Inventor: Husain SHEKHANI, Duluth, GA (US)

(73) Assignee: Bausch + Lomb Ireland Limited, Dublin (IE)

(21) Appl. No.: 17/229,149

(22) Filed: Apr. 13, 2021

(51) Int. Cl. A61F 9/007

(52) U.S. Cl. CPC A61F 9/00781

(57) ABSTRACT

A phacoemulsification handpiece for ocular surgery includes a housing including a grip for holding the phacoemulsification handpiece, a needle extending from the housing for removing ocular material from an eye, and a horn connected with the needle. The phacoemulsification handpiece also includes a driving circuit configured to vibrate the horn according to a multi-harmonic driving signal, to generate non-sinusoidal ultrasonic motion of a tip of the needle.

### FIELD

The present disclosure relates to systems and methods for generating resonant non-sinusoidal drive signals for phacoemulsification surgery devices.

### BACKGROUND

This section provides background information related to the present disclosure which is not necessarily prior art.

A cataract clouds the natural lens of the eye, which includes mostly water and protein. Over time, these proteins may clump together to obscure the lens. This is generally corrected by removing the cataract lens and replacing it with a clear lens implant, such as through phacoemulsification eye surgery.

Phacoemulsification is a surgery technique on an eye where the internal lens is emulsified with a phacoemulsification (e.g., phaco) needle tip, which is driven to vibrate ultrasonically by an ultrasonic mechanism in the phaco surgical handpiece. The ultrasonic vibration of the phaco needle may create a temperature rise of the needle, which can occur essentially instantaneously. The emulsified lens material (which is mostly fluid) is aspirated from the eye through the phaco needle and replaced with an irrigation fluid (e.g., a balanced salt solution (BSS), etc.). Intraocular pressure (IOP) is maintained in the eye while the phaco needle is aspirating ocular material from the eye by continuously infusing saline solution into the eye. The constant replenishment of fluids in the eye is important to avoid collapse of the anterior chamber of the eye. The irrigation fluid also cools the heating effects of the vibrating phaco needle, thus preventing burning of eye tissue at the incision site. Occasionally, large chunks of ocular material clog the phaco needle, which interrupts the aspiration flow and in turn causes interruption in the irrigation flow.

### SUMMARY

This section provides a general summary of the disclosure, and is not a comprehensive disclosure of its full scope or all of its features.

According to one aspect of the present disclosure, a phacoemulsification handpiece for ocular surgery includes a housing including a grip for holding the phacoemulsification handpiece, a needle extending from the housing for removing ocular material from an eye, and a horn connected with the needle. The phacoemulsification handpiece also includes a driving circuit configured to vibrate the horn according to a multi-harmonic driving signal, to generate non-sinusoidal ultrasonic motion of a tip of the needle.

According to another aspect of the present disclosure, a phacoemulsification handpiece for ocular surgery includes a housing including a grip for holding the phacoemulsification handpiece, a needle extending from the housing for removing ocular material from an eye, and a horn connected with the needle. The phacoemulsification handpiece also includes a driving circuit configured to vibrate the horn according to a driving signal including multiple rectangular waves, to generate non-sinusoidal ultrasonic motion of a tip of the needle.

According to yet another example embodiment of the present disclosure, a method of driving a needle of an ocular handpiece for ocular surgery is disclosed. The handpiece includes a housing having a grip for holding the ocular handpiece, with the needle extending from the housing for removing ocular material from an eye, and a horn connected with the needle. The method includes combining multiple harmonic frequencies to generate a multi-harmonic driving signal, and vibrating the horn according to the multi-harmonic driving signal to generate non-sinusoidal ultrasonic motion of a tip of the needle.

Further aspects and areas of applicability will become apparent from the description provided herein. It should be understood that various aspects of this disclosure may be implemented individually or in combination with one or more other aspects. It should also be understood that the description and specific examples herein are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1A is a waveform illustrating an example sinusoidal driving signal for a needle tip of a phacoemulsification handpiece, according to the prior art.

FIG. 1B is a waveform illustrating an example sinusoidal displacement of a needle tip of a phacoemulsification handpiece driven by the signal of FIG. 1A.

FIG. 2 is a block diagram of an ocular surgical handpiece, according to an aspect of the present disclosure.

FIG. 3A illustrates a combination of a first harmonic waveform and a second harmonic waveform.

FIG. 3B illustrates a resulting waveform according to the combination of waveforms in FIG. 3A.

FIG. 4A illustrates an example rectangular wave for generating a non-sinusoidal driving signal, according to another aspect of the present disclosure.

FIG. 4B illustrates example harmonic frequencies of rectangular waves driven at fifty percent duty ratios.

FIG. 4C illustrates example harmonics frequencies of rectangular waves driven at twenty percent duty ratios.

Corresponding reference numerals indicate corresponding parts or features throughout the several views of the drawings.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

In known phaco handpieces, the needle tip is ultrasonically vibrated longitudinally (e.g., like a jack-hammer). For example, a piezo stack or magneto-resistive circuit vibrates a horn and needle to move the tip of the needle back and forth in line with a central axis of the handpiece. It is also known to construct various vibration assemblies that move the needle tip side-to-side (e.g., off-center weighting, orientation of piezo pieces, bent needles, etc.), to provide torsional/twisting movement (e.g., using helical grooves in the horn), or to provide elliptical movement.

Some of these non-longitudinal movements are created mostly at resonant vibration frequencies separate from the longitudinal resonant frequency. Therefore, some techniques allow a user to switch, during surgery, from mostly or only longitudinal vibration to break up the cataract, to mostly or only an alternate vibration motion, or possibly an intermediate ratio of combined vibration motions.

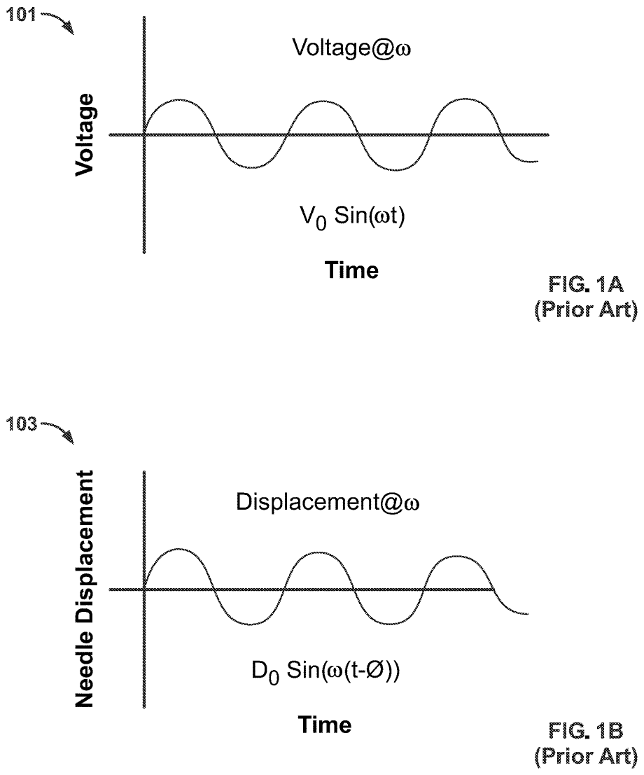

Longitudinal vibration generally provides the most effective motion to break-up and emulsify cataract tissue. For example, a traditional longitudinal phaco driving signal 101 is illustrated in FIG. 1A. The sinusoidal function V_{∘} sin(ωt) includes a voltage that oscillates at a sinusoidal frequency ω. FIG. 1B illustrates a displacement 103 of the needle based on the sinusoidal driving signal. As shown in FIG. 1B, the needle is displaced at the sinusoidal frequency ω, according to a function D_{∘} sin(ω(t−0)).

Disclosed herein are example embodiments of driving an ultrasonic phacoemulsification handpiece in order to create non-sinusoidal motion at a tip of a needle of the phacoemulsification handpiece. When using traditional sinusoidal driving signals (e.g., one frequency at a time, two frequencies where each frequency has one or more common nodal points along the handpiece to minimize vibration to the user, switching between two drive frequencies with one or more common modal points), there is no ability to adjust the operation of the device other than adjusting the amplitude. As described further herein, example embodiments that use non-sinusoidal motion may allow for adjustment of amplitude ratio, phase, etc. of a multi-harmonic driving signal to alter the needle tip displacement waveform to achieve advantages over sinusoidal driving signals.

For example, if different harmonics are excited, their comparative amplitude ratio and phase difference will determine the shape of the resultant displacement waveform. A resultant non-sinusoidal displacement waveform may reduce or alter the fluid flow pattern in the eye due to the ultrasonic vibrations. For example, the non-sinusoidal ultrasonic motion of the needle tip may bring fluid toward the needle in a manner similar to ultrasonic torsion, may help reduce occlusions, etc.

The non-sinusoidal ultrasonic motion may alter the physical mechanisms responsible for phacoemulsification. For example, the phacoemulsification mechanisms may be altered based on the type of cataract (e.g., a soft lens or a very hard lens). A user can select a displacement waveform that targets cauterization, mechanical impact, etc. The non-sinusoidal ultrasonic motion may provide more effective emulsification, may produce less heat during surgery to reduce a chance of burns, etc.

A mixture of first and second harmonics may produce an asymmetric needle tip displacement, similar to a saw tooth pattern. Changing the relative phases of the first and second harmonics may change the direction of the asymmetry of the needle tip displacement.

In some embodiments, rectangular waves may be used to produce the first and second harmonics (e.g., by driving the rectangular wave at a fundamental frequency and stimulating a duty ratio of other than fifty percent). By varying the duty ratio above or below fifty percent, the direction of the asymmetry of the needle tip displacement may change.

Practically, changing the relative phases or duty ratio may result in changing a direction of increased velocity of the needle tip between a direction away from a transducer that drives the needle, and a direction towards the transducer. Additionally, ultrasonic saw tooth motion of the needle may enable higher tip velocities using less power, while maintaining continuous excitation.

Although some embodiments are described herein with reference to a phacoemulsification handpiece, other embodiments may include other suitable handpieces for other ocular surgeries, such as a vitrectomy handpiece, etc.

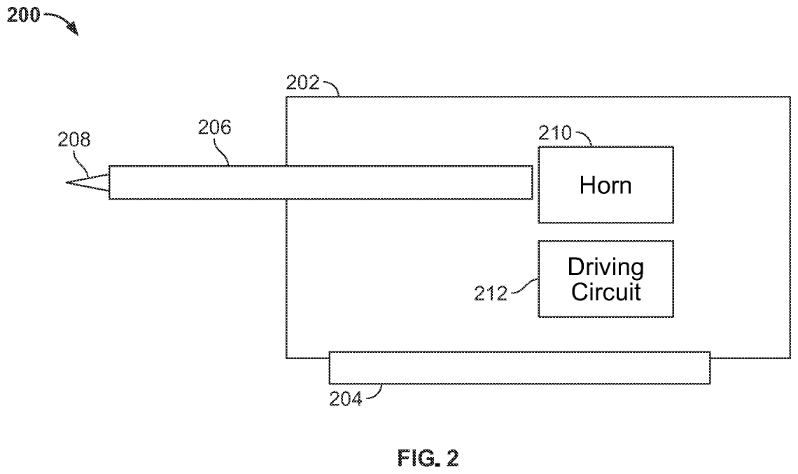

A handpiece for ocular surgery (e.g., phacoemulsification, etc.) according to one example embodiment of the present disclosure is illustrated in FIG. 2 and indicated generally by reference number 200. The handpiece 200 includes a housing 202 having a grip 204 for holding the handpiece 200.

The handpiece 200 also includes a needle 206 (e.g., an aspiration needle) extending from the housing 202 for removing ocular material from an eye, a horn 210 connected with the needle 206, and a driving circuit 212. The driving circuit 212 is configured to vibrate the horn 210 according to a multi-harmonic driving signal, to generate non-sinusoidal ultrasonic motion of a tip 208 of the needle 206.

The driving circuit 212 may include any suitable circuit, processor, etc. for driving the horn 210. For example, the driving circuit 212 may include a piezoelectric element stack, a magneto-resistive circuit, etc., which is driven with a multi-harmonic, non-sinusoidal voltage signal.

In some embodiments, the handpiece 200 may be connected to an aspiration tube for receiving the ocular material removed from the eye by the needle 206, an irrigation tube (which may be integrally formed with the aspiration tube) for supplying irrigation fluid to the surgical handpiece 200, and a power cord for suppling power to the driving circuit 212, etc.

For example, a console may include a source of balanced salt solution (BSS). The irrigation tube may be adapted to supply the irrigation fluid from the source of BSS to the handpiece 200 during the ocular surgery, to maintain an inter-ocular pressure of the eye as the ocular material is aspirated from the eye through the surgical handpiece 200.

The console may also include an aspiration reservoir, where the aspiration tube is connected between the handpiece 200 and the aspiration reservoir (e.g., to supply fluid that has been aspirated from the eye from the handpiece 200 to the aspiration reservoir). An aspiration pump may be housed in the console to generate a vacuum pressure for suctioning fluid through the aspiration tube from the handpiece 200 to the aspiration reservoir.

For example, during phacoemulsification surgery the internal lens of the eye is emulsified with the phacoemulsification needle 206, which is driven to vibrate ultrasonically by the driving circuit 212 and the horn 210 in the handpiece 200. The ultrasonic vibration of the needle 206 significantly increases the temperature of the needle 206. The emulsified lens material is aspirated from the eye through the needle 206 and the aspiration tube to the aspiration reservoir in the console, and replaced with an irrigation fluid from the BSS source via the irrigation tube.

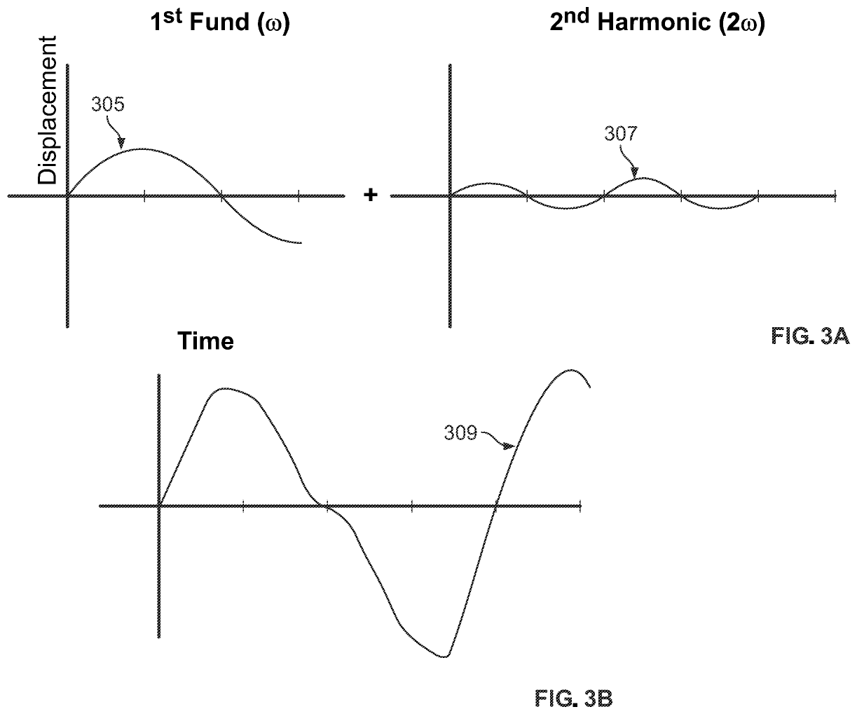

FIGS. 3A and 3B illustrate a multi-harmonic driving signal 309 that includes a combination of a first fundamental frequency signal 305 (e.g., a first harmonic) and a second harmonic frequency signal 307. For example, FIG. 3A illustrates a signal that creates a needle displacement at the first fundamental frequency ω, which is added to a signal that creates a needle displacement at a second harmonic frequency 2ω.

Adding the first and second harmonic signals 305 and 307 together results in a multi-harmonic, non-sinusoidal driving signal 309 as shown in FIG. 3B. The driving signal 309 may have a waveform that is similar to a saw tooth waveform pattern.

Adding the first and second harmonic signals 305 and 307 together may be considered as mode mixing. In some embodiments, the driving signal 309 may include a full amplitude of the first harmonic signal 305, and a half amplitude of the second harmonic signal 307. In other embodiments, different relative amplitudes may be used, the harmonics may have the same amplitude, more than two harmonics may be combined, the driving signal may include harmonics other than the first and second, etc.

The driving circuit 212 may be configured to adjust a phase of the first harmonic relative to a phase of the second harmonic to change an orientation of the waveform, may be configured to adjust an amplitude ratio between the first and second harmonics, etc. Therefore, the driving signal 309 may include a combination of the first and second harmonics according to a specified amplitude ratio, according to a specified phase difference, etc.

The driving circuit 212 may be configured to adjust an amplitude of one of the harmonic frequencies relative to an amplitude of another of the harmonic frequencies to generate the non-sinusoidal motion of the tip 208 of the needle 206. Similarly, the driving circuit 212 may be configured to adjust a phase of one the harmonic frequencies relative to a phase of another of the harmonic frequencies to generate the non-sinusoidal motion of the tip 208 of the needle 206.

As described above, the driving circuit 212 may be configured to mix the harmonic frequencies to produce an asymmetric saw tooth displacement of the tip 208 of the needle 206. For example, adjusting a phase of the harmonic frequencies relative to one another may change a direction of the asymmetry of the displacement of the tip 208 of the needle 206.

The horn 210 may be positioned to move the tip 208 of the needle 206 back and forth along a central axis of the handpiece 200. Portions of the driving signal 309 having smaller slopes may correspond to lower velocity movement of the needle 206, while portions of the driving signal having greater slopes may correspond to higher velocity movement of the needle 206.

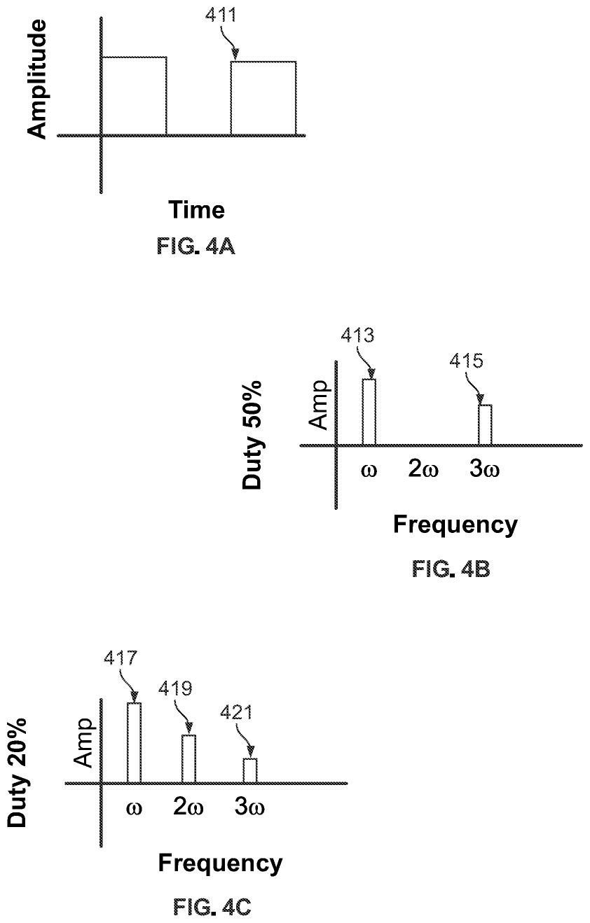

FIG. 4A illustrates a rectangular wave 411 that may be used to drive the horn 210, according to another example embodiment of the present disclosure. For example, the driving circuit 212 may be configured to vibrate the horn 210 according to a driving signal including multiple rectangular waves 411, to generate non-sinusoidal ultrasonic motion of the tip 208 of the needle 206.

As shown in FIG. 4B, the multiple rectangular waves may include a first rectangular wave having a fundamental frequency 413 (e.g., a first harmonic) and a second rectangular wave having a second harmonic frequency 415. For example, the first harmonic 413 may have a frequency of ω, while the second harmonic frequency 415 has a frequency of 3ω.

In FIG. 4B, the rectangular waves are driven at a duty cycle/ratio of fifty percent. FIG. 4C illustrates example harmonic frequencies when the rectangular waves are driven at a twenty percent duty cycle/ratio. In FIG. 4C, the signal includes a first harmonic 417 at a frequency of ω, a second harmonic 419 at a frequency of 2ω and a third harmonic 421 at a frequency of 3ω. In other embodiments, more or less harmonics may be used, harmonics other than the first, second or third may be used, etc.

The driving signal may include a duty ratio other than fifty percent for at least one of multiple rectangular waves, to generate the non-sinusoidal motion of the tip 208 of the needle 210. For example, the driving circuit 212 may be configured to adjust the duty ratio above or below fifty percent to change a direction of asymmetry of the non-sinusoidal motion of the tip 208 of the needle 210.

The driving circuit may be configured to adjust the duty ratio above or below fifty percent to change a direction of increased velocity of the tip 208 of the needle 206, between an increased velocity towards the driving circuit 212 and an increased velocity away from the driving circuit 212. Although FIG. 4C illustrates a duty ratio of twenty percent, other embodiments may use any suitable duty ratio that is more or less than twenty percent.

As described herein, the handpiece 200 and the driving circuit 212 may include a microprocessor, microcontroller, integrated circuit, digital signal processor, etc., which may include memory. The handpiece 200 and the driving circuit 212 may be configured to perform (e.g., operable to perform, etc.) any processes for controlling displacement of the needle 206 using any suitable hardware and/or software implementation. For example, the handpiece 200 and the driving circuit 212 may execute computer-executable instructions stored in a memory, may include one or more logic gates, control circuitry, etc.

According to another example embodiment of the present disclosure, a method of driving a needle of an ocular handpiece for ocular surgery is disclosed. The handpiece includes a housing having a grip for holding the ocular handpiece, with the needle extending from the housing for removing ocular material from an eye, and a horn connected with the needle.

The method includes combining multiple harmonic frequencies to generate a multi-harmonic driving signal, and vibrating the horn according to the multi-harmonic driving signal to generate non-sinusoidal ultrasonic motion of a tip of the needle. Combining the multiple harmonic frequencies may include combining at least two harmonic frequencies according to a specified amplitude ratio or phase difference.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A phacoemulsification handpiece for ocular surgery, the phacoemulsification handpiece comprising:
a housing including a grip for holding the phacoemulsification handpiece;
a needle extending from the housing for removing ocular material from an eye;
a horn connected with the needle; and
a driving circuit configured to vibrate the horn according to a multi-harmonic driving signal, to generate non-sinusoidal ultrasonic motion of a tip of the needle.

2. The phacoemulsification handpiece of claim 1, wherein the driving circuit comprises piezoelectric element stack.

3. The phacoemulsification handpiece of claim 1, wherein the driving circuit comprises a magneto-resistive circuit.

4. The phacoemulsification handpiece of claim 1, wherein the multi-harmonic driving signal comprises a waveform that includes a combination of a first fundamental frequency and a second harmonic frequency.

5. The phacoemulsification handpiece of claim 4, wherein the waveform includes a full amplitude of the first fundamental frequency and a half amplitude of the second harmonic frequency.

6. The phacoemulsification handpiece of claim 4, wherein the driving circuit is configured to adjust a phase of the first fundamental frequency relative to the second harmonic frequency to change an orientation of the waveform.

7. The phacoemulsification handpiece of claim 1, wherein the multi-harmonic driving signal comprises a waveform that includes a combination of at least two harmonic frequencies according to a specified amplitude ratio or phase difference.

8. The phacoemulsification handpiece of claim 7, wherein the driving circuit is configured to adjust an amplitude of one of the at least two harmonic frequencies relative to an amplitude of another one of the at least two harmonic frequencies to generate the non-sinusoidal ultrasonic motion of the tip of the needle.

9. The phacoemulsification handpiece of claim 7, wherein the driving circuit is configured to adjust a phase of one of the at least two harmonic frequencies relative to a phase of another one of the at least two harmonic frequencies to generate the non-sinusoidal ultrasonic motion of the tip of the needle.

10. The phacoemulsification handpiece of claim 7, wherein the driving circuit is configured to mix the at least two harmonic frequencies to produce an asymmetric saw tooth displacement of the tip of the needle.

11. The phacoemulsification handpiece of claim 10, wherein the driving circuit is configured to adjust a phase of the at least two harmonic frequencies relative to one another to change a direction of asymmetry of displacement of the tip of the needle.

12. The phacoemulsification handpiece of claim 1, wherein the horn is positioned to move the tip of the needle back and forth longitudinally along a central axis of the handpiece.

13. A phacoemulsification handpiece for ocular surgery, the phacoemulsification handpiece comprising:
a housing including a grip for holding the phacoemulsification handpiece;
a needle extending from the housing for removing ocular material from an eye;
a horn connected with the needle; and
a driving circuit configured to vibrate the horn according to a driving signal including multiple rectangular waves, to generate non-sinusoidal ultrasonic motion of a tip of the needle.

14. The phacoemulsification handpiece of claim 13, wherein the multiple rectangular waves comprise a first rectangular wave having a fundamental frequency and a second rectangular wave having a harmonic frequency.

15. The phacoemulsification handpiece of claim 13, wherein the driving signal includes a duty ratio other than fifty percent for at least one of the multiple rectangular waves, to generate the non-sinusoidal ultrasonic motion of the tip of the needle.

16. The phacoemulsification handpiece of claim 15, wherein the driving circuit is configured to adjust the duty ratio above or below fifty percent to change a direction of asymmetry of the non-sinusoidal ultrasonic motion of the tip of the needle.

17. The phacoemulsification handpiece of claim 15, wherein the driving circuit is configured to adjust the duty ratio above or below fifty percent to change a direction of increased velocity of the tip of the needle between increased velocity towards the driving circuit and increased velocity away from the driving circuit.

18. The phacoemulsification handpiece of claim 15, wherein the duty ratio comprises a twenty percent duty ratio.

19. A method of driving a needle of a handpiece for ocular surgery, the handpiece including a housing having a grip for holding the ocular handpiece, the needle extending from the housing for removing ocular material from an eye, a horn connected with the needle, the method comprising:
combining multiple harmonic frequencies to generate a multi-harmonic driving signal; and
vibrating the horn according to the multi-harmonic driving signal to generate non-sinusoidal ultrasonic motion of a tip of the needle.

20. The method of claim 19, wherein combining the multiple harmonic frequencies includes combining at least two harmonic frequencies according to a specified amplitude ratio or phase difference.
